(19) United States
(12) Patent Application Publication
(10) Pub. No.: US 2008/0057134 A1
(43) Pub. Date: Mar. 6, 2008

(54) Antifungal compositions and method

(75) Inventor: Joseph J. Crudden, Hudson, NH (US)

Correspondence Address: EDWARD K. WELCH II;IP&L Solution, 4558 Ashton Court, Naples, FL, 34112, US

(73) Assignee: AgION Technologies, Inc.

(21) Appl. No.: 11/897,698

(22) Filed: Aug. 31, 2007

Related U.S. Application Data
(60) Provisional application No. 60/841,517, filed on Aug. 31, 2006.

(51) Int. Cl. A61K 31/045; A61K 31/13; A61K 31/17; A61K 31/18; A61K 31/195; A61K 31/38; A61K 31/415; A61K 31/42; A61K 31/425; A61K 31/535; A61K 31/66; A61K 31/675; A61K 31/69; A61K 33/24; A61K 33/28; A61K 33/30; A61K 33/32; A61K 33/34; A61K 33/36; A61K 33/38

(52) U.S. Cl. 424/617; 424/600; 424/618; 424/620; 424/630; 424/639; 424/641; 424/644; 424/646; 424/649; 424/650; 424/651; 424/652; 424/653; 424/654; 424/655; 514/231.2; 514/235.8; 514/256; 514/367; 514/372; 514/374; 514/394; 514/417; 514/438; 514/561; 514/567; 514/579; 514/588; 514/601; 514/64; 514/724; 514/75; 514/93

(57) ABSTRACT

Efficacious antifungal compositions are prepared comprising a mixture of a traditional fungicide and an ion-exchange type antimicrobial agent.

### RELATED APPLICATION

This patent application claims the benefit of previously filed U.S. Provisional Patent Application No. 60/841,517 entitled “Antifungal Compositions and Method” filed on Aug. 31, 2006, which is hereby incorporated herein in its entirety by reference.

### FIELD OF THE INVENTION

The present invention relates to antifungal compositions, coatings containing the same and methods of using such compositions and coatings in preventing the growth of molds and, more specifically, in conjunction therewith, the preservation of wood and wood products. More specifically, the present invention is directed to synergistic combinations of certain antifungal agents and ion-exchange type antimicrobial agents, especially those comprising a combination of silver ion and copper ion sources or a single source of both silver and copper ions.

### BACKGROUND OF THE INVENTION

For centuries man has identified and used natural agents and minerals to combat fungus, especially in crops and in the preservation of wood. For the past half-century and more, many major chemical, now bioscience, companies have focused on the synthesis of fungicides for use in battling fungi, the number one cause of crop damage and loss worldwide. Even today, the thought of spending millions in designing, synthesizing and testing potential fungicides for crop protection is short change compared to the economic gain should a new, broad spectrum and efficacious fungicide be developed. Though the use of fungicides has not been restricted solely to crop protection, until recently fungicides have found limited use outside of the agricultural area other than as mildew preventative additives for paints and caulks.

The ongoing evolution in building design and construction in every market segment, including commercial, industrial, warehouse, residential, etc., has seen a marked and frightening proliferation in the appearance of mold contamination of buildings. Unlike buildings of the first half of the 20^{th} century, the buildings that followed and of today tend to be ‘closed’ boxes: windows don't open and doors and trucking bays are designed to keep out the elements. The drafty old houses and multifamily and apartment buildings of yesteryear have been replaced by air-tight, highly insulated boxes which rely upon air conditioners to alter the internal atmosphere. In commercial, warehouse and industrial buildings the open construction of days past when pipes and conduits hung, suspended from the ceiling are gone in favor of false ceilings or defined conduit/pipe corridors where air flow is minimal at best. All of this has led to the construction of incubators for microbes and fungi: dead spaces where the air is stagnant and moisture is allowed to build, whether as a result of ambient moisture, condensation or leaks. Indeed, with this closed construction leaks in conduits or pipes or even structural leaks take longer to appear, if they appear at all, and while the immediate leak may be found, water that has seeped into wall spaces, etc., oftentimes goes undetected or unheeded.

Further compounding the problem of building design is building materials. Instead of hardwoods, more and more building products are made from soft woods which are much more amenable to fungal and microbe growth. More problematic is the use of manufactured wood products, e.g., plywood, and synthetic building materials such as pressed wood products, laminate products and other cellulosic products that integrate glues and like: materials that provide a superior nutrient base for the growth of fungi and bacteria.

With all these warm, humid and stagnant air spaces or environments and a ready source of nutrients, it is no wonder that there is an alarming growth in the appearance of mold contamination in houses and apartments as well as commercial, industrial, warehouse and other buildings. Similarly, it is no coincidence that medical and environmental health professionals have coined the phrase ‘sick building syndrome’ for the myriad of symptoms including respiratory distress, asthma or allergic type reactions, etc., manifested by people working and/or residing in these buildings.

While the removal of contaminated building materials and the cleaning of contaminated areas with proper sanitizers like bleach are successful in eradicating the immediate problem, they do not prevent the re-contamination of that area. More importantly, they do nothing to address the spores given off by the mold that have traveled on air currents or in the air conditioning system to other areas of the building. Thus, there is an immediate and growing need for agents that provide long lasting, continuous inhibition to the growth of mold in buildings. The scope, strength and urgency of this need are further evidenced by the plethora of lawsuits and insurance claims that have been filed relative to mold-in-buildings, sick building syndrome and the like.

Although numerous chemical impregnation preservatives have long been employed to preserve wood and wood products, it has not been financially viable to use such material in all aspects of construction. Furthermore, many of such early antifungal and preservative treatments are inappropriate for indoor applications due to health and safety concerns. Thus, these materials have primarily been relegated to outdoor exposure applications. Even then, many of these materials have since been banned and others still in use are undergoing strict scrutiny due to extreme human and environmental health and safety concerns.

In an effort to address this ever growing problem several entrepreneurial companies have more recently introduced products, notably coatings, that builders can apply to the building materials, for example, framing, basement walls, etc. that are said to prevent the growth of mold. Many of these products are based on traditional fungicides; however, most traditional fungicides have limited use, attacking a pre-existing problem rather than preventing a future or subsequent mold contamination. Furthermore, due to toxicological and environmental health and safety concerns, many fungicides, for example those based on mercury, arsenic, and the like, are not suitable for use in buildings. While certain antimicrobial agents typically employed for consumer oriented applications to prevent the growth of bacteria have found utility in anti-fungal or anti-mold applications, their efficacy against molds is low.

Thus, there is a need for fungicides, including wood preservative products, that are efficacious against molds in buildings. Additionally, there is a need for fungicides that are suitable for use in environments where exposure to humans, pets and/or food or food stock products is likely. More importantly, there is a need for fungicide compositions which provide efficacious anti-fungal, specifically anti-mold, activity for extended periods of time.

Similarly, there is a continual and growing need for wood preservative products that are highly efficacious with minimal, if any, human and environmental heath and safety concerns. This is of particular importance with the substantial decline in hardwoods, many of which are naturally resistant to rot and insects, and the increase reliance upon soft, fast growing woods that are readily susceptible to attack and degradation. The importance is further manifest and enhanced by the growing use of manufactured wood products that contain glues and other organic materials that provide fertile feeding grounds for and promote the growth of molds.

Finally, there is a growing need for improved antifungal agents in the agricultural domain. This need is multifold stemming from an increase in the occurrence of active resistance in a number of microorganisms as well as growing concerns to the ever-increasing loading and bioaccumulation of biocides in the environment and in the food chain, right up to us humans. Thus, any means by which the overall amount of biocides, including fungicides, can be reduced without a loss in bioefficacy would be of tremendous benefit and value.

### SUMMARY OF THE INVENTION

In accordance with the teaching of the present invention there are provided novel antifungal compositions comprising traditional antifungal agents in combination with one or more ion-exchange type antimicrobial agent. In particular there are provided combinations of antifungal agents based upon thiazolones, especially isothiazolones, or borates, especially perborate, in combination with one or more ion-exchange type antimicrobial agents comprising an ion-exchange carrier having ion-exchanged therewith silver ions, copper ions or a combination of silver and copper ions.

In a second aspect, the present invention pertains to antifungal coating compositions comprising a polymer binder or matrix material and the combination of an antifungal agent and one or more ion-exchange type antimicrobial agents. In particular, it relates to coating compositions comprising a non-brittle, preferably hydrophilic, binder or polymer matrix material and the combination of the antifungal agent and the one or more ion-exchange type antimicrobial agents.

In yet another aspect, the present invention relates to wood preservatives and a method of preserving wood wherein the inventive combination of borate fungicides and ion-exchange type antimicrobial agents are applied to the surface of and/or impregnated into wood, wood stock and wood products.

Finally, the present invention also pertains to a method of inhibiting the growth of molds and mildew in buildings and building components which method comprises the step of treating the materials, preferably the stock materials, used in the fabrication or construction of a building, or portions thereof, with an antifungal composition comprising an antifungal agent and one or more ion-exchange type antimicrobial agents. Alternatively, the present invention pertains to a method of inhibiting the growth of molds and mildew in buildings and building components which method comprises the step of treating the erected elements of the building with an antifungal coating composition comprising a traditional antifungal agent and one or more ion-exchange type antimicrobial agents. In each of these alternative methods, it is preferred that the treatment method comprises the application of an coating composition comprising a binder or other polymer matrix forming material and the antifungal composition to the building materials or elements or the erected components of the building.

### DETAILED DESCRIPTION OF THE INVENTION

All patent applications, patents, patent publications, and literature references cited in this specification, whether referenced as such, are hereby incorporated by reference in their entirety. In the case of inconsistencies, the present description, including definitions, is intended to control.

The present invention provides various compositions and methods for rendering surfaces, particularly building surfaces, resistant to the growth and proliferation of molds and mildew. When used herein and in the appended claims, the term “resistant” means that no visible growth, as seen by the unaided human eye, is detected on the treated substrate after 28 days incubation on NSA agar.

Antifungal agents useful in the practice of the present invention vary widely; though, at the present time, some, if not most, may not be registered for building applications. Nevertheless, many different chemistries or families of antifungal agents may be used and are well known. However, care must be taken to avoid those antifungal agents that will or are known to readily and irreversibly, or substantially so, react with silver and/or copper ions. Such a reaction or strong sequestration of the silver and/or copper ions of the antimicrobial agents by the fungicide in normal use will likely diminish the bioefficacy thereof. Additionally, since the compositions of the present invention are used in buildings and building components as well as in various articles of manufacture, all of which involve or are likely to involve direct human contact or contact through materials that become airborne due to their vapor pressures, it is best to avoid those antifungal agents that are notably toxic and/or create concerns relative to environmental health and safety. For example, it is best to avoid the use of mercurial or arsenate based fungicides due to the highly toxic nature of mercury and arsenic, respectively. On the other hand, fungicides that, in accordance with the present invention, are used at levels sufficiently below those at which toxicity would be of concern may also be employed. Indeed, one beneficial aspect of the present invention is the finding that certain fungicides, especially the borates and certain organic fungicides and/or their metal salts, provide a synergy with the ion-exchange antimicrobial agents, thereby enabling one to use less fungicide for the same or better degree of efficacy.

As noted, suitable antifungal agents are well known and commercially available. They vary widely and belong to many different chemical classes including the aliphatic nitrogen fungicides; the amide fungicides including the amide, acyl amino acid, anilide, benzanilide, furanilide, sulfonanilide, bezamide, furamide, phenyl sulfamide, sulfonamide, valinamide fungicides; the antibiotic fungicides including the strobilurin fungicides; the aromatic fungicides including the chloroneb, chlorothalonil, dichlorobenil, dichloran, and PCNB fungicides; the benzimidazole fungicides; the benzothiazole fungicides; the bridged diphenyl fungicides; the carbamate fungicides, including the carbamate, benzimidazoylcarbamate and carbanilate fungicides; the conazole fungicides including the imidazole and triazole fungicides; the copper fungicides; the dicarboximide fungicides including dicarboximide, dichlorophenyl dicarboximide and phthalimide fungicides; the dinitrophenol fungicides; the dithiocarbamate fungicides including dithiocarbamate, cyclic dithiocarbamate, and polymeric dithiocarbamate fungicides; the imidazole fungicides; the inorganic mercuric and organomercury fungicides; the morpholine fungicides; the organophosphorus fungicides; the organotin fungicides; the oxathiin fungicides; the oxazole fungicides; the polysulfide fungicides; the pyrazole fungicides; the pyridine fungicides; the pyrimidine fungicides; the pyrrole fungicides; the quinoline fungicides; the quinone fungicides; the quinoxaline fungicides; the thiazole fungicides; the thiocarbamate fungicides; the thiophene fungicides; the triazine fungicides; the triazole fungicides, the urea fungicides; the borate fungicides; and the like. These and more fungicides, as well as specific examples of each, are set forth in the PAN Pesticide Database, www.pesticideinfo.org. and in The Compendium of Common Names—Fungicides, www.alanwoods.net/pesticides, both of which are incorporated herein by reference. Preferred classes of fungicides include the azoles (especially the triazoles, imidazoles and myclobutanils), substituted benzenes, borates, carboxamides, chlorothalonils, dithiocarbamates, isothiazolones, strobilurins, and the like. Notwithstanding their inadvertent inclusion in the classes or subclasses of fungicides above, those fungicides which, irrespective of the level used, adversely interact with the ion-exchange antimicrobial agent whereby the antifungal performance is significantly compromised are not desirable and should be avoided. While an insignificant interference with the antifungal performance may be tolerated, especially where there is a need for both antifungal and antimicrobial performance, preferably there will be no adverse effect on the antifungal performance of the fungicide at the level employed.

Those skilled in the art will readily recognize that the classes and subclasses of fungicides mentioned above embrace a plethora of actual fungicide compounds and commercial fungicide compositions. Exemplary fungicides include methyl-thiophanate (sold under the tradenames Ztopsin M, Clearly's 3336), triflumizole (sold under the tradenames Procure and Terraguard), ethylene(bis)dithiocarbamate (sold under the tradenames Mancozeb and Manzeb), myclobutanil (sold under the tradenames Immunox, Eagle, Nova and Rally), chlorothalonil (sold under the tradename Daconil), tebuconazole (sold under the tradename Folicur), azoxystrobin (sold under the tradenames Abound, Quandris, Heritage), trifuozystrobin (sold under the tradenames Flint, Gem and Compass), pyraclostrobin (sold under the tradenames Cabrio and headline), etridiazole (sold under the tradenames Trubana and Koban) and dichloro octyl isothiazolone (sold under the tradenames Rozone and Rocima). When using a formulated fungicide, care should be taken to ensure that other components, carriers, adjuvants, etc. contained in such formulated products do not adversely interfere with and/or react with the antimicrobial metal ions of the antimicrobial additive. Some degree of chelation/sequestering can be accommodated so long as the bond is reversible in use and/or the extent thereof is not sufficient to adversely affect the bioefficacy of the ion-exchange antimicrobial agent. Additionally, while many of the formulated fungicides are available in solution/emulsion/suspension or the like, where a liquid carrier is present, it is preferably based on aqueous, aqueous-based, or water soluble carriers as opposed to water insoluble carriers such as the phenyl ethers and the like. Especially preferred fungicides are the triazoles, myclobutanils, imidazoles, borates, isothiazoles, dithiocarbamates, and strobilurins.

Perhaps the largest class of suitable fungicides are the azoles, a class which embraces several subclasses including the diazoles, triazoles, myclobutanils, and imidazoles, benzimidazoles, triadiazoles, benzothiadiazoles, thiodiazoles, imidazolidones, and thiazolones, in particular the triazoles. The latter includes, for example, cyproconazole, epoxiconazole, fluquinconazole, flusilazole, flutriafol, metconazole, prochloraz, propiconazole, prothioconaxole and tebuconazole. Other suitable azoles include, for example, terrazole, fenbuconazole, bromoconazole, cyazofamid, difenoconazole, imazalil, imazalil sulfate, myclobutanil, tetraconazole, and the like.

The isothiazolone fungicides are also an important class of suitable fungicides. These include the octhilinone; Kathon 886; 5-chloro-2-methyl-4-isothizolin-3-one; 1,2-benzisothiazolin-3-one; 2-butyl-,2-benzisothiazolin-3-one; 2-methyl-4-isothizolin-3-one; 2-methyl-4-isothizolin-3-one calcium chloride complex; and the like.

Yet another suitable class of fungicides include the strobilurins or strobins. Exemplary strobins include azoxystrobin, dimoxystrobin, fluoxastrobin, kresoxim-methyl, metominostrobin, picoxystrobin, pyraclostrobin, and trifloxystrobin.

As noted, the dithiocarbamate fungicides represent yet another beneficial class of fungicides. Exemplary dithiocarbamates include cufraneb, ferbam, mancozeb, manganous dimethyldithiocarbamate, metam potassium, metiram, nebam, potassium dimethyldithiocarbamate, sodium dimethyldithiocarbamate, ziram and the like.

Finally, yet another especially beneficial class of fungicides useful in the practice of the present invention, particularly in wood preservation and in preventing mold growth on wood and wood products, are the borates, including the perborates, particularly the sodium borates. Such materials are well known in the industry and include, for example, boric acid, sodium borate, borax, sodium tetraborate decahydrate, sodium tetraborate pentahydrate, disodium octaborate tetrahydrate, disodium octoborate, sodium metaborate, sodium perborate, sodium perborate tetrahydrate, sodium pentaborate decahydrate, and the like.

The second component of the fungicide composition according to the present invention is the ion-exchange type antimicrobial agent. These agents typically comprise an inorganic ion-exchange carrier and one or more antimicrobial metals and/or metal ions, most preferably one or more antimicrobial metal ions. Suitable antimicrobial metals and metal ions include, but are not limited to, silver, copper, zinc, gold, mercury, tin, lead, iron, cobalt, nickel, manganese, arsenic, antimony, bismuth, barium, cadmium, chromium and thallium. Metal ions of silver, copper, zinc, and gold or combinations thereof are preferred because they are considered safe for human contact uses. Silver ions, alone or in combination with copper or zinc or both, are more preferred due to the fact that they have the highest ratio of efficacy to toxicity, i.e., high efficacy to low toxicity.

The inorganic ion-exchange carrier is preferably an ion-exchange type ceramic particle wherein antimicrobial metal ions have been exchanged (replaced) for other non-antimicrobially effective ions in the ceramic particles or a combination of the foregoing with an antimicrobial metal salt. Suitable ceramic particles include, but are not limited to zeolites, hydroxyapatite, zirconium phosphates and other ion-exchange ceramics, and come in many forms and types, including natural and synthetic forms. For example, the broad term “zeolite” refers to aluminosilicates having a three dimensional skeletal structure that is represented by the formula: XM₂/nO—Al₂O₃—YSiO₂-ZH₂O wherein M represents an ion-exchangeable ion, generally a monovalent or divalent metal ion; n represents the atomic valency of the (metal) ion; X and Y represent coefficients of metal oxide and silica, respectively; and Z represents the number of water of crystallization. Examples of such zeolites include A-type zeolites, X-type zeolites, Y-type zeolites, T-type zeolites, high-silica zeolites, sodalite, mordenite, analcite, clinoptilolite, chabazite and erionite. The present invention is not restricted to use of these specific zeolites.

Ion-exchange type antimicrobial agents are widely available and are becoming well known. Additionally, they are widely discussed in the patent literature. For example, hydroxyapatite particles containing antimicrobial metals are described in, e.g., U.S. Pat. No. 5,009,898. Zirconium phosphates containing antimicrobial metals are described in, e.g., U.S. Pat. No. 5,296,238; U.S. Pat. No. 5,441,717 and U.S. Pat. No. 5,405,644. More preferably, the antimicrobial agent is an antimicrobial zeolite containing ion-exchanged antimicrobial metal ions. Antimicrobial zeolites, including the antimicrobial zeolites disclosed in U.S. Pat. No. 4,911,898; U.S. Pat. No. 4,911,899 and U.S. Pat. No. 4,938,958, are well known and may be prepared for use in the present invention using known methods. Though much of the discussion of the ion-exchange antimicrobial agents will be focused on the zeolites, those skilled in the art will recognize that the discussion, as well as the ranges, parameters, etc. mentioned, are equally applicable to and readily translatable to the other ion-exchange carriers. Furthermore, since all of these antimicrobial agents are commercially available and described in the patent literature, as mentioned above, their composition and the like are known to those skilled in the art.

The ion-exchange antimicrobial agents may incorporate most any antimicrobial metal ions, including those mentioned above. Most commonly, though, silver ions or silver ions in combination with zinc and/or copper ions are used. The amount of antimicrobial metal ion is generally in the range of from about 0.1 to about 25 wt %, preferably from about 0.3 to about 20 wt %, most preferably from about 2, to about 10 wt %, based upon 100% total weight of ceramic carrier. Where other antimicrobial metal ions are present, the makeup will be from about 0.1 to about 15 wt % of silver ions and from about 0.1 to about 15 wt % of copper and/or zinc ions. These ion-exchange type antimicrobial agents may also have incorporated therein ion-exchanged ammonium ions for improved color stability. If present, the ammonium ions may be present at a level of up to about 20 wt % of the carrier; however, it is desirable to limit the content of ammonium ions to about 0.5 to about 2.5 wt %.

Especially preferred ion-exchange antimicrobial agents are the antimicrobial zeolites available from AgION Technologies, Inc., of Wakefield, Mass., USA including, but not limited to the following product numbers: AW10D (0.6% by weight of silver ion-exchanged in Type A zeolite particles having a mean average diameter of about 3μ), AG10N and LG10N (2.5% by weight of silver ion-exchanged in Type A zeolite particles having a mean average diameter of about 3μ and 10μ, respectively); AJ10D (2.5% silver, 14% by weight zinc, and between 0.5% and 2.5% by weight ammonium ion-exchanged therein in Type A zeolite having a mean average diameter of about 3μ); AK10D (5.0% by weight of silver ion-exchanged in Type A zeolite particles having a mean average diameter of about 3μ) and AC10D (6.0% by weight of copper and 3.5% by weight silver ion-exchanged in Type A zeolite particles having a mean average diameter of about 3μ).

Generally speaking, ion-exchange type antimicrobial agents are prepared by an ion-exchange reaction in which non-antimicrobial ions such as sodium ions, calcium ions, potassium ions and/or iron ions, present in the carrier particles are partially or wholly replaced with antimicrobial metal ions. Suitable antimicrobial metal ions include those mentioned above. Similarly, as noted previously, the preferred antimicrobial metal ions employed in the ion-exchange type antimicrobial agents are silver, copper and zinc ions or combinations thereof, and most preferably silver ions alone or together with one or both of the others. For example, a combination of silver and copper ions provides both the antibacterial properties of the silver ions and the antifungal properties of the copper ions. Thus, one is able to tailor the antimicrobial agent by selection of specific metal ions and combinations thereof to be incorporated into the ion-exchange carrier particles for particular end-use applications.

The ion-exchange type antimicrobial agent to be used in the practice of the present invention can be used in its neat form or it may be encapsulated as described in United States Published Patent Application No. US2003-0118664 A1 (U.S. Ser. No. 10/032,372 filed Dec. 21, 2001 by Trogolo et al.), which is incorporated herein by reference. Generally speaking, the encapsulated antimicrobial agent is in the form of microcapsules or particles that comprise either a single particle or, most preferably, a plurality (several to several hundred or more) of particles of the antimicrobial agent encapsulated within a hydrophilic polymer. The encapsulated antimicrobial agent may be of many shapes and may deform somewhat during processing of the coating. Generally, the encapsulated antimicrobial agent will be in the form of particles having a low aspect ratio, for example, on the order of from 1 to about 4, preferably from 1 to about 2, most preferably from 1 to about 1.5. However, it is also contemplated that microcapsules may be of a high aspect ratio as taught in United States Published Patent Application No. US2003-0118658 A1 (U.S. Ser. No. 10/032,370 filed Dec. 21, 2001 by Trogolo et al), also incorporated herein by reference. These high aspect ratio microcapsules are typically in the shape of flakes and fibers whose aspect ratio is up to 100 or more, but typically is less than about 30.

The hydrophilic polymers suitable for use in encapsulating the antimicrobial agent are those that can absorb sufficient water to enable the encapsulated particle to exhibit good antimicrobial behavior, i.e., to allow for the migration and release of the antimicrobial active agent through and from the encapsulating polymer matrix. These polymers are characterized as having water absorption at equilibrium of at least about 2% by weight, preferably at least about 5% by weight, more preferably at least about 20% by weight, as measured by ASTM D570. Especially suitable hydrophilic polymers include those having water contents at equilibrium of from about 50 and to about 150% by weight.

The encapsulating hydrophilic polymers, hereinafter oftentimes referred to as the encapsulant, are typically comprised of substantial quantities of monomers having polar groups associated with them, such that the overall polymeric composition is rendered hydrophilic. The polar groups can be incorporated into the polymer main chain as in for example polyesters, polyurethanes, polyethers or polyamides. Optionally the polar groups can be pendant to the main chain as in for example, polyvinyl alcohol, polyacrylic acids or as in ionomers such as Surlyn®. Surlyn® is available from Dupont and is the random copolymer poly(ethylene-co-methacrylic acid) wherein some or all of the methacrylic acid units are neutralized with a suitable cation, commonly Na⁺ or Zn². While not being limited by way of theory, it is believed that the inclusion of polar groups allows water to more readily permeate the polymer and consequently, to allow slow transport of the metal ion through the encapsulating polymer layer. Such encapsulants may be thermoplastic or they may be thermoset or cross-linked.

A number of specific hydrophilic polymers suitable for use as the encapsulant include, for example, (poly)hydroxyethyl methacrylate, (poly)hydroxypropyl methacrylate, (poly)glycerol methacrylate, copolymers of hydroxyethyl methacrylate and/or methacrylic acid, polyacrylamide, hyaluronan, polysaccharides, polylactic acid, copolymers of lactic acid, (poly)vinyl pyrrolidone, polyamides such as Nylon 6,6, Nylon 4,6 and Nylon 6,12, cellulosics, polyureas, polyurethanes and certain polyesters containing a high percentage (at least about 10% by weight, preferably at least about 25% by weight or more) of polyalkylene oxide.

The hydrophilic polymer may be a copolymer containing at least a substantial amount of at least one or more of the above-mentioned hydrophilic monomers, including, for example, styrene/methacrylic acid/hydroxyethyl methacrylate copolymers, styrene/methacrylic acid/hydroxypropyl methacrylate copolymers, methylmethacrylate/methacrylic acid copolymers, ethyl methacrylate/styrene/-methacrylic acid copolymers and ethyl methacrylate/methyl methacrylate/styrene/methacrylic acid copolymers, copolymers based upon the cellulosics, and copolymers which utilize vinylpyrrolidone monomers, among numerous others, especially copolymers of n-vinylpyrrolidone and polymethylmethacrylate.

Other encapsulants include polyvinyl acetate, polyvinyl alcohol, and copolymers of polyvinyl alcohol and polyvinylacetate, polyvinylchloride, copolymers of polyvinylacetate and polyvinylchloride and hydroxyl-modified vinyl chloride/vinyl acetate copolymers.

Polyurethanes containing a high percentage (at least about 10% by weight, preferably at least about 25% by weight or more) of polyalkylene oxide are especially useful in this invention.

Preferably the encapsulating hydrophilic polymer is chosen from polyhydroxyethyl methacrylate, polyacrylamide, polyvinylpyrrolidinone, polyurea, polysaccharides, polylactic acid, poly(meth) acrylic acid, polyurethane and copolymers thereof. More preferably, the hydrophilic polymer is hydrophilic polyurethane, such as the TECOPHILIC® polyurethane sold by Thermedics of Woburn, Mass. or a lightly cross-linked polymer based on n-vinylpyrrolidone and methylmethacrylate sold under the trade designation AEP Polymers by I H Polymeric Products Limited of Kent, England.

While the encapsulated antimicrobial agent may be in the form of individually encapsulated antimicrobial particles having a coating thickness of up to 15μ, more typically and preferably, they are in the form of larger microcapsules containing multiple antimicrobial particles, especially of the ion-exchange type. The latter typically comprise from about 5 wt % to about 65 wt %, preferably from about 20 wt % to about 50 wt % of the antimicrobial agent based on the total weight of the encapsulated antimicrobial agent. Although the latter microcapsules may have a mean average diameter of up to and over 2000μ, for use in the present invention their size will be much smaller, generally they will have a mean average diameter of up to about 300μ, preferably from about 30μ to about 200μ, most preferably from about 50μ to about 150μ. Of course smaller or larger microcapsules can be used depending upon the size of the activated carbon particles.

Encapsulated antimicrobial agents are especially useful where the binder is not a hydrophilic material. This is because the transport mechanism by which the ion-exchange type antimicrobial agents work is reliant upon a liquid medium, preferably water, bringing ions to the antimicrobial particle to exchange with and thereby release the antimicrobial metal ions. Thus, unless there are pathways through the binder or the binder is a hydrophilic material, at least a portion of the surface of the ion-exchange type antimicrobial agent must be exposed in order for a given particle of the antimicrobial agent to be effective. With the former, such pathways may be naturally occurring, well defined pathways or channels as in the case of porous polymers and inorganic binders or they may be in the form of molecular sized pores that exist between molecules and/or polymer chains in the case of thin layers of the binder, i.e., nano- or angstrom scale.

The encapsulated antimicrobial agents enhance bioefficacy on two fronts. First, the significantly larger particle size of the encapsulated antimicrobial agents increases the likelihood that any one particle will have an exposed surface or touch or in be close proximity to the surface of the binder. Secondly, because the entire amount of the antimicrobial active within a given particle of the encapsulated antimicrobial agent is accessible, those particles having a plurality of particles of the antimicrobial agent incorporated therein serve as large reservoirs of the antimicrobial active, i.e., the antimicrobial metal ions. Furthermore, because the degree of hydrophilicity controls, in part, the release rate of the antimicrobial metal ions, these materials also provide for greater longevity combined with the excellent release. And, where the binder is itself a hydrophilic polymer, the use of the encapsulated antimicrobial agent allows one to further regulate the release of the antimicrobial agent by encapsulating the antimicrobial agent with a hydrophilic polymer having a different degree of hydrophilicity. For example, if the encapsulating material is of a lower hydrophilicity than the binder, it will serve to slow the release of the antimicrobial ions.

The amounts of the antifungal active and the ion-exchange type antimicrobial agent present in the novel antifungal compositions will vary widely depending upon the fungi or molds to be addressed as well as the specific selection of the respective active agents. Generally speaking the weight ratio of the antifungal active to the ion-exchange type antimicrobial agent in the antifungal compositions of the present invention will be from about 0.005:1 to 3:1, preferably from about 0.05:1 to about 1:1. Of course, these ratios will vary markedly depending upon the specific antifungal agent chosen. For example, a fungicidal composition wherein the fungicide active is chlorothalonil (e.g., Daconil supplied as a spray concentrate, at 29.6% active, by The Ortho Group, Columbus, Ohio) will be used at a level of 1% fluid ounces per 4 gallons of water; whereas the fungicide active myclobutanil (e.g., Immunox supplied as a spray concentrate, at 1.55% active by the Spectrum group, a division of United Industries Corporation, St Louis Mo.) will typically be used at a level of one fluid ounce per gallon of water.

Another factor indirectly influencing the amount of the antifungal active and the antimicrobial agent as well as the weight ratio of the antifungal active to the ion-exchange antimicrobial agent, at least from a commercial standpoint, is the rate of application or the amount by which the composition is allowed to be used in a given end-use application. Specifically, antifungal and antimicrobial agents, and the use thereof, are typically regulated by one or more governmental authorities or agencies: for example, in the United States, non-medical/non-veterinarian uses, are regulated by the US Environmental Protection Agency. These regulations not only specify the end-uses to which these materials may be applied but also specify the loadings or rate of application, i.e., how much may be used/applied for a given area or mass. Thus, while higher levels may be successfully employed from a purely efficacious standpoint, they may not be employed from a practical standpoint, at least not until the labels are amended to allow the higher use levels. In the meantime; the product labels for the active agents to be employed will help guide one in selecting the appropriate compositional make-up to be used.

Regardless, though it is important for the combinations of fungicide and antimicrobial agent to not adversely affect the bioefficacy of each other, it is most desirable that these combinations provide a synergy with respect to their bioefficacy, especially with respect to the antifungal activity. This synergy has two key attributes. First, for a given level of the active agent, it enables one to obtain a higher level of bioefficacy than is attainable with each agent by itself. Perhaps more importantly, though, especially given the environmental health and safety concerns with their uses, it enables one to attain the same level, if not a higher level, of bioefficacy with less, preferably much less, of either or both ingredients. Indeed, as shown below in the examples, the combinations of actives of the present invention oftentimes provide excellent bioefficacy even though each of the actives is present at a level for which no or little bioefficacy is shown when used on their own.

The antifungal compositions of the present invention may be employed as is or are preferably incorporated into various thermoset and/or thermoplastic coating or binder compositions, especially paints and other like film forming materials. Coatings/binder compositions are typically of two types, those comprising or containing a resin or polymer material, either in solution or suspended in a liquid carrier (e.g., a dispersion, suspension, colloid or emulsion), which forms a film upon evaporation or loss of the solvent or carrier, as appropriate, and those which are free or substantially free of solvents or carriers and involve at least one physical transformation of the coating/binder material as applied to the substrate, either from a liquid or flowable 100% solids material to a solid or semi-solid film or layer of a polymer material (i.e., curable coatings) or from a particulate solid material to a substantially uniform film or layer of the solid material through heat (powder coatings). The curable coatings are perhaps the most diverse and may take a number of forms in and of themselves. For example, they may comprise one-part systems that cure or set upon exposure to certain environmental conditions, e.g., heat, light, moisture. Alternatively, they may comprise two- or more-part systems that are essentially shelf stable as long as the parts remain isolated from one another but cure or become curable upon mixing of the two or more parts, e.g., coatings that contain a catalyst in one part and an initiator in another.

Although the discussion herein refers to film forming coatings and binders, it is also understood that such coating and binder compositions may be such that discrete domains of the “cured” coating or binder material rather than a film is formed. Here the polymer or polymer forming materials of the uncured coating/binder become associated with particles of the fungicide and/or ion-exchange type antimicrobial and, upon application and cure, bind individual or groups of individual fungicide particles and/or ion-exchange type antimicrobial agent particles to the substrate surface.

The coatings of the present invention may be single layer or multi-layered systems wherein each layer may have originated from a single or multi-part coating composition and provides different physical properties and/or bioefficacy benefits. A preferred multilayered coating system is one wherein a hydrophilic coating is applied as a topcoat over a non-hydrophilic coating. Such systems are disclosed in, e.g., Trogolo et. al. US 20050287375, which is incorporated herein by reference. When incorporating the aforementioned antifungal compositions, these systems provide excellent short term or immediate bioactivity as well as long term durability and antifungal/antimicrobial activity. Selection of the coating both in terms of its composition, its form and, if appropriate, cure modality, will depend upon the specific substrate to be treated, the method of application, and the environmental and use conditions to which it will be exposed and, in following, the physical properties desired of the coating material itself. Since conventional coatings may be modified for use in the practice of the present invention, those skilled in the art will select the appropriate coating for their given application.

The chemistry or formulation of the thermoset or thermoplastic coating compositions vary widely and are selected based on the desired physical properties of the coating compositions, the mode of application (e.g., solution based, curable 100% solids or powder coating), the pot life (if applicable), the cure mechanism (i.e., heat, UV light, moisture, etc.), and the environmental conditions to which they are exposed in use. Typically, in the case of thermoset coatings the choice of polymer or polymerizable components is based on the cure method and pot life as well as the adhesion, wear, and appearance characteristics or properties. In the case of thermoplastic coatings, selection of the thermoplastic polymer is based on the solvent needed and/or the ease of application, especially as powder coatings, as well as their adhesion, wear and appearance characteristics or properties.

Suitable thermoplastic polymers include, but are not limited to, polypropylene, polyethylene, polystyrene, ABS, SAN, polybutylene terephthalate, polyethylene terephthalate, nylon 6, nylon 6,6, nylon 4,6, nylon 12, polyvinylchloride, polyurethanes, silicone polymers, polycarbonates, polyphenylene ethers, polyamides, polyethylene vinyl acetate, polyethylene ethyl acrylate, polylactic acid, polysaccharides, polytetrafluoroethylene, polyimides, polysulfones, and a variety of other thermoplastic polymers and copolymers.

Suitable thermoset or cross-linkable coatings include, but are not limited to, phenolic resins, urea resins, epoxy resins, including epoxy-novolak resins, polyesters, epoxy polyesters, acrylics, acrylic and methacrylic esters, polyurethanes, acrylic or urethane fortified waxes and a variety of other thermoset or thermosettable polymers and copolymers. Thermoset coating systems based on epoxy resins, whether 100% solids or aqueous dispersions/latexes, are especially preferred due to their excellent adhesive properties and durability. Suitable epoxy resin systems include those sold by Corro-Shield of Rosemont, Ill. as well as Burke Industrial Coatings of Vancouver, Wash.

In certain applications, especially those where the coating or binder is not exposed to wear or erosion or not exposed at all, e.g., wall studding, unexposed surfaces of wall board, and the like, it may be preferable to employ binders and coatings based on hydrophilic polymers. Hydrophilic materials are desirable due to the fact that all of the antifungal and antimicrobial agent in the hydrophilic polymer is available for providing antimicrobial activity. Polymers which have little or no hydrophilic characteristics do not allow for the migration and ion-exchange of the antimicrobial metal ions of the carrier particles encased or entombed in the polymer material as no water transport mechanism exists within and through the polymer. Here the relatively poor physical and durability properties of the hydrophilic polymers are irrelevant; whereas the excellent release characteristics are exceptional and especially beneficial.

The hydrophilic polymers may be either thermoset (i.e., curable thermosetting or cross-linking polymer compositions) or thermoplastic compositions. Suitable hydrophilic polymers include those previously mentioned and discussed at length above with respect to the encapsulation of the antimicrobial agent. Hydrophilic polymer coatings include coatings comprising any of the aforementioned hydrophilic polymers used in making the encapsulated antiviral agents, as discussed above. Alternatively, coatings of certain traditional non-hydrophilic polymers may me made hydrophilic by blending a hydrophilic polymer with a non-hydrophilic polymer and/or cross-linkable coating polymer precursor. A preferred blend is made by using a supporting polymer comprising a plurality of functional moieties capable of undergoing crosslinking reactions, said supporting polymer being soluble in or emulsified in an aqueous based medium; and a hydrophilic polymer, said hydrophilic polymer being associated with the supporting polymer. The ratio of the hydrophilic to non-hydrophilic and/or cross-linkable polymer depends on the hydrophilicity of the hydrophilic polymer and the desired hydrophilicity of the resultant blend. Especially preferred hydrophilic binders are those based poly(meth)acrylates and poly (meth)acrylic acids or on cross-linked polyurethanes described in, e.g., U.S. Pat. No. 6,238,799 and U.S. Pat. No. 6,866,936 and available from Surface Solutions Laboratories of Carlisle, Mass.

The antifungal binder or coating compositions of the present invention may be prepared in accordance with any conventional method of coating preparation. For example, the antifungal composition comprising the antifungal agent and the ion-exchange type antimicrobial agent may be added to the coating formulation as it is being prepared or following its preparation. Alternatively, especially in the case of dissolved coatings or dispersions, suspensions and the like, it may be incorporated into the polymer or polymer forming material prior to incorporation thereof in the solvent. In yet another alternative method, antifungal compositions may be applied subsequent to the application of the coating or binder systems. In the latter instance, the aforementioned antifungal composition is either dusted or sprinkled over the coated surface prior to cure of the binder coating or it may be applied in a mist of an activator or activator solution which effectuates cure (including evaporation of a solvent, if applicable) of the binder coating. Incorporation into the liquid compositions immediately prior to application or subsequent to application is especially preferred where there is any concern that one or both active components of the antifungal composition may adversely interact with the components of the coating composition during production and/or long-term storage.

In the case of powder coatings, the antifungal compositions may be blended with the preformed powder coating particles or they may be incorporated into the pre-mix for the same, thereby dispersing the antimicrobial agent into the powder coating particles themselves. Alternatively, the components of the antifungal compositions may be fused to the powder coating particles, especially by heat fusion. Fusing avoids the separation and settling of the antifungal components from the powder coating particles, especially as a result of the shaking and vibrations experienced during transportation and application preparation. Also, fusing avoids the entombment of the agent in the polymer particle, and thus coating.

As noted previously, coatings produced in accordance with the teaching of the present invention may comprise a single layer or two or more layers, each of which incorporates the antifungal compositions. Single layer coatings are preferred due to their simplicity of application; however, as noted above, most coating applications do not allow for the use of hydrophilic polymers and, therefore, there is concern for the antifungal compositions components, especially the ion-exchange type antimicrobial agents, contained within the coating and below the surface thereof. This concern may only be temporary in the case of coated surfaces that are subject to wear during use, especially floors. Alternatively, even those coatings, as well as all non-hydrophilic coatings where skinning over is a concern, can be activated by quickly eroding the surface layer of polymer coating. Depending upon the physical properties of the coatings, such may be achieved simply by buffing and/or lightly sanding the surface. Yet another alternative would be to employ hydrophilic polymer encapsulated antimicrobial agents as discussed above.

Generally speaking, the amount of antifungal composition incorporated into or employed in conjunction with the coating or binder is typically from about 3 wt % to about 50 wt %, preferably from about 5 wt % to about 30 wt %, most preferably from about 10 wt % to about 25 wt %, based on the total weight of the cured coating or binder, i.e., the solids or solid forming components. In the case of encapsulated antimicrobial agents, the wt % of the antimicrobial agent is based on the amount of antimicrobial agent itself (including the carrier), exclusive of the encapsulant.

In addition, the coating or binder compositions may also employ other compounds in combination with antifungal compositions for the purpose of imparting better and/or more immediate efficacy. For example, certain salts, such as sodium salts, including sodium nitrate, may be used in combination with the ion-exchange antimicrobial agent to enhance the initial release of the antimicrobial agent by providing a ready source of cations to exchange with and, thereby, enable the release of, the antimicrobial metal ions. Such additional components are desirable so long as they are biocompatible and do not interfere with the bioefficacy of the antimicrobial agent.

Finally, the coating formulations may also contain other additives such as UV or thermal stabilizers, adhesion promoters, dyes or pigments, leveling agents, fillers and solvents. In the case of multilayered coatings, such additives may be added to one or both layers depending upon the nature of the additive and the performance required. The specific additives to be use and the amount by which they can be used will depend upon the end use application and the choice of the polymer. Generally speaking, though, the selection and level of incorporation will be consistent with the directions of their manufacturers and/or known to those skilled in the art.

Antifungal coating compositions formed in accordance with the present invention may be applied by any of the methods known in the art, including spraying, brushing, rolling, printing, dipping and mold coating, powder coating, etc. The selection and thickness of the coating or coatings, in the case of multi-layered systems, can vary widely and depends upon the application requirements and limitations. For example, a high wear environment may require at thicker coating, especially one of good durability and/or wear resistance. The thickness of the coating, or the base coat in the case of multi-layered coatings, may also be a function of life of the substrate to which it is applied or, if the coating is periodically refinished or removed and replaced, the intended life of the coating itself. Generally, the thickness is the same as would be used for such coating compositions in the absence of the antifungal composition. Since, in practice, the antifungal composition may be added to commercially available coating compositions, typically the thickness and rate of application will be as recommended by the manufacturer of the same. However, given the aforementioned issues with components, especially the ion-exchange antimicrobial agents, that lie below the surface of non-hydrophilic coating or are not mobile within the coatings, the additional factors come into consideration as discussed below.

When the coating or binder composition is a non-hydrophilic, especially a skin forming non-hydrophilic, composition, it is especially preferred that the thickness of the coating is, at most, slightly thicker than, but preferably the same as or less than, the average particle size or, in the case of encapsulated antimicrobial agents, the effective particle size of the ion-exchange type antimicrobial agent and/or that a higher loading of the antimicrobial agent is employed so as to increase its concentration at or near the surface. Average particle sizes of slightly less than the thickness of the coating are possible since the distribution of particles will still provide a good number of particles in excess of the coating thickness and the coating thickness itself oftentimes varies across the surface of the substrate to which it is applied. Thus, the goal is to ensure that an adequate number of particles of the antimicrobial agent have not skinned over so that a sufficient level of antimicrobial metal ion release is capable without having to wear away or remove the skin. In this respect one would want for at least about 30%, preferably at least about 40%, of the particles of the antimicrobial agent to have a diameter of equal to or less than the thickness of the coating. Though one could add greater quantities of antimicrobial agents whose average particle size is more than a micron or so less than the thickness of the coating, such would not be economical, especially in relatively low cost applications.

Preferred coatings for use in the practice of the present invention, whether as the sole coat or as a base or topcoat, will be such that the particles of the antimicrobial agent do not readily settle in the coating formulation once applied. Settling has the same effect as skinning as the coating material flows over the top of the particles as they settle in the composition. Thus, coatings having a high viscosity, e.g., typical of house paint or higher, or manifesting thixotropic behavior are especially preferred. In essence, it is especially desirable that the viscosity of the coating composition be such that, following application, the coating composition cures before any significant settling has occurred, particularly where the thickness of the coating as applied to the substrate is to be greater than the particle size of the antimicrobial agent. Another way of achieving such high concentrations of antimicrobial agent at the surface is the dusting of the wet, uncured, coating material with the antifungal composition following the application of the coating to the substrate surface but before cure of the same, as mentioned earlier.

While the foregoing discussion has focused on the ion-exchange antimicrobial agent component of the antifungal compositions, the controlling particle size relative to coating thickness will be the lesser of the ion-exchange antimicrobial agents or the antifungal agents in the case of antifungal agents that do not migrate through and within the polymer matrix of the coating or binder compositions.

The versatility and ease of use of the coating compositions of the present invention make them especially desirable and provide for a nearly endless list of end-use applications. They may be applied to any of a number of surfaces or articles of manufacture, regardless of their manufacture, i.e., whether they are composed of metal, plastic, wood, glass, paper, fabric/textile, etc., or structure, i.e., film, sheet, fabric, fiber, strip, pole, pipe, etc. These compositions may be applied to stock materials (e.g., fabric) and/or to finished goods and articles of manufacture (e.g., cushions, upholstered products, curtains, etc.) with the selection of the specific coating matrix being dependent, in part, upon the surface to be coated, the application method, and the conditions to which it is exposed so as to ensure sufficient surface wetting and adhesion. Such characteristics are known in the art and/or are supplied by manufacturers of various base coating materials into which the antifungal compositions are incorporated.

The antifungal coating and binder compositions are especially suited for treatment of exposed and unexposed surfaces of buildings and other structures susceptible to mold growth. For example, conduits, especially ventilation conduits, and open studding and framework may be treated/coated before the conduits are installed/sealed and the walls closed, respectively. Similarly, the exposed and unexposed surfaces of wallboard and other wall/ceiling forming materials may be treated during manufacture or prior to installation. Similarly, shingles, tiles and other roof forming materials may be treated prior to or following installation to ward off mold growth.

In following, the antifungal coating and binder compositions are especially suited for retrofit applications where a building, structure or article of manufacture is treated subsequent to their initial use, especially where the building, structure or article of manufacture has already experienced fungal growth. This may be especially desirable for articles of manufacture to be placed into surface in high temperature and humidity environments, e.g., in Florida, Mississippi and Louisiana, or in defined environments subject to such conditions, e.g., greenhouses, breweries, bakeries, laundries, and the like, etc. While the coating or binder compositions could be applied to the contaminated surface to attack the fungal growth, it is preferred to first clean the surfaces to remove all or substantially all fungal growth before applying the antifungal coating or binder composition: indeed, such may be a requirement to ensure good adhesion of the coating material to the substrate surface. The subsequent application of the antifungal coating or binder will attack any remaining fungal growth and prevent or at least significantly hinder the reoccurrence thereof. The application of the antifungal coatings and binders is most appropriate in association with the making of repairs/renovation following water damage/leaks.

The antifungal compositions of the present invention may also be directly incorporated into the materials from which articles of manufacture are made. Specifically, the antifungal compositions may be directly compounded into various resins and polymer compositions, especially thermoplastic compositions, which are subsequently molded, extruded, pultruded, etc. into a finished good or a stock material used in making a finished good or substrate. Similarly, they may be incorporated into the precursor materials for various composite and thermoset compositions concurrent with or prior to their molding or forming process to make finished goods or stock materials. As with the coatings, there is little by way of limitation as to the end-use applications to which the antifungal thermoplastic and thermoset compositions of the present invention may be applied.

Since the vast majority of thermoplastics are not hydrophilic and, in any event, hydrophilic materials have very limited applications, various specialized plastic forming and processing methods may be employed in order to minimize the amount of the antifungal composition actives that are not accessible and, thus, ineffective until exposed. For example, films, sheet and articles of manufacture may be made by co-extrusion methods whereby the outer exposed surface(s) carry the antifungal composition or at least those actives which are unable to migrate through the polymer matrix while the inner or center layers or a surface where antifungal activity is not needed, is free of the antifungal composition. Other methods include over-molding, rotational molding, and the like where only the exposed polymer material contains the antifungal composition. Similarly, one may prepare laminate structures where the exposed laminate surface incorporates the antifungal composition but the under layers or substrate to which they are applied or adhered do not.

As noted above, the antifungal compositions may be incorporated into most any plastic or polymer material, whether thermoplastic or thermoset, including silicones and the like. Exemplary thermoplastics include, but are certainly not limited to, any of those mentioned previously with respect to thermoplastic coating materials, including, or as well as, polyesters, polyolefins, polyetheresters, polyetherimides, polyimides, polyamides, polyphenylene ethers, polystyrenes, ABS, polycarbonates, thermoplastic elastomers (TPEs), polyvinylchloride, polyvinylethers, polyvinylacetates, polyacrylates and poly(meth)acrylates, and the like. Exemplary thermoset materials include, but are not limited to those mentioned previously with respect to the thermoset coating materials including, or as well as, thermosetting polyesters, epoxy resins, thermosetting polyurethanes, alkyds, phenol-formaldehyde resins, urea-formaldehyde resins and the like.

The antifungal compositions are incorporated into the polymer materials by any known method suitable for the given antifungal composition and the selected polymer materials. For example, melt blending and solution blending are especially suited for thermoplastic materials, the latter especially where the antifungal composition contains a component that is heat sensitive, especially at or near the melt temperatures of the polymer. Otherwise, especially for thermoset materials, the antifungal composition can be incorporated into one or more of the prepolymers or other materials used in forming the polymer materials prior to polymerization thereof.

The antifungal compositions may also be incorporated into any number of inorganic materials, especially those employed in building construction, like cements, mortar, grout, plaster, and the like, or in the manufacture of building materials, such as ceramics and cements for tiles, especially roofing tiles, or in the molding of various articles/goods such as planters, statutes, and the like.

The amount of antifungal composition to be incorporated into the polymer or inorganic materials is typically from about 1.0 to about 30 wt %, preferably from about 3.0 to 20 wt %, based on the total weight of the polymer or inorganic composition into which it is incorporated.

The following examples are presented as demonstrating the unexpected synergy of the combination of fungicide and ion-exchange type antimicrobial agent in preventing the growth of fungus, especially molds. These examples are merely illustrative of the invention and are not to be deemed limiting thereof. Those skilled in the art will recognize many variations that are within the spirit of the invention and scope of the claims.

### EXAMPLES 1-4 AND COMPARATIVE EXAMPLES CE1-CE10

Two sets of experiments were conducted to evaluate the efficacy of a series of fungicidal compositions, the formulations of which are set forth in Table 1, against Aspergillus niger. Each set of experiments was identical with the exception that the second set of experiments was evaluated at an elevated pH and in the presence of surfactant to reduce surface tension and improve wetting.

The fungicidal coatings were prepared by uniformly dispersing the active agents identified in Table 1, in 10 ml of water. Thereafter, 60 ml of an aqueous emulsion of a vinyl acetate-acrylic copolymer (Flexbond® 325 emulsion from Air Products, Inc, of Allentown, Pa., USA) was added to the so formed dispersion and the mixture thoroughly mixed. Water was then added, with mixing, to bring the total weight to 100 grams.

As noted above, the second set of examples was conducted at elevated pH. This was achieved by adding an alkaline anionic surfactant solution to each fungicidal coating composition at a ratio of 1 ml surfactant solution/100 ml coating. The alkaline surfactant solution was prepared by combining 5 ml of a pH 10 carbonate buffer (Potassium Carbonate/Potassium borate/Potassium hydroxide buffer from Fisher Scientific); one sodium hydroxide chip; 5 ml of an aqueous surfactant solution containing 10% sodium lauroyl sarcosinate (L30 from Hampshire Chemical Corp of Nashua, N.H., USA), 30% lauryl dimethylamine-oxide (LDAO from Stepan Company of Northfield, Ill. 60093, USA); and water: the latter added to bring the total mixture to 30 ml in volume.

Test specimens for performing fungus testing were prepared by applying 1 ml of each coating to Whatman filter paper and spreading the coating over an area of slightly greater than 1 square inch. The coating was allowed to dry for several days to a hard flexible tack-free film. A 1 square inch specimen was then cut from the coated paper for testing.

The efficacy of the fungicidal coatings was evaluated by direct inoculation fungal testing using a modified ASTM G21 testing protocol. Testing was conducted on two different test media plates, NSA—nutrient salt agar, a medium nutrient agar, and PDA—potato dextrose agar, a high nutrient agar. In each test, the test specimen was placed on the agar media, coating side up. Thereafter, 0.4 ml of a lyophilized culture suspension of Aspergillus niger was aseptically applied to the surface of each specimen and spread evenly over the surface with a sterile loop. The agar plates were then incubated at 28° C. for 28 days and then observed for fungal growth. The results were reported in accordance with the following classifications:

| | | Numerical |
|---|---|---|
| Specimen Coverage | Classification | Classification |
| 0% | No growth | 0 |
| <10% | Trace growth | 1 |
| 10%-<30% | Slight growth | 2 |
| 30%-<60% | Moderate growth | 3 |
| 60%-100% | Heavy growth | 4 |

The study demonstrates the bioefficacy performance of the individual components as well as the combination of the ion-exchange type antimicrobial agent with certain compounds having fungicidal properties in preventing/inhibiting the growth Aspergillus niger. Specifically, the active materials/agents evaluated in this set of experiments included a silver/copper zeolite antimicrobial agent (AgION AC10D), two commercial formulations of the dichloro octyl isothiazolone fungicide—one aqueous based (Rocima) and the other non-aqueous based (Rozone), and picrotone olamine (Octopirox—monoethanolamine salt of hydroxymethyl trimethylpentyl pyridone), another antimicrobial agent that has been shown to have some antifungal activity. As seen in Table 1, the silver/copper zeolite antimicrobial agent had essentially no antifungal properties on its own; whereas both isothiazolone fungicides and picrotone olamine provided excellent to good antifungal properties. When the silver/copper zeolite was combined with the isothiazolones, the antifungal performance was essentially unchanged, or, surprisingly, enhanced. Specifically, the combination of the silver/copper zeolite and the aqueous based isothiazolone fungicide (Rocima) manifested an unexpected synergy, even when the concentration of each active was reduced by fifty percent (Examples 1 and 2). These results were consistent in both test systems, even in the highly nutritious PDA agar. Perhaps more importantly, is the fact that these results were attained using loadings of the fungicide and the silver/copper zeolite at levels below their traditional/recommended use levels.

On the other hand, when the silver/copper zeolite was combined with picrotone olamine, the relatively good antifungal performance of the picrotone olamine was compromised by the presence of the silver/copper zeolite, even in NSA, a moderate nutrient agar. Specifically, as seen in Comparative Examples CE6 through CE10, those specimens treated with the combination of the two antimicrobial agents manifested a higher degree of fungal growth than those treated with picrotone olamine alone. In light of these, it is surprising that such an adverse effect on antifungal bioefficacy was not manifest in the traditional antifungal agents,

TABLE 1

| | EXAMPLES | | | | | | | | | | | | | | |
|---|---|---|---|---|---|---|---|---|---|---|---|---|---|---|---|
| | CE1 | CE2 | 1 | 2 | CE3 | 3 | CE4 | 4 | CE5 | 5 | CE6 | CE7 | CE8 | CE9 | CE10 |
| ACTIVE | | | | | | | | | | | | | | | |
| AGENTS | | | | | | | | | | | | | | | |
| AgION AC10D¹ | 1 | 0 | 1 | 0.5 | 0 | 1 | 0 | 1 | 0 | 1 | — | 0 | 1 | 0 | 1 |
| Rocima 200² | — | 0.2 | 0.2 | 0.1 | 0.5 | 0.5 | — | — | — | — | — | — | — | — | — |
| Rozone³ | — | — | — | — | — | — | 0.2 | 0.2 | 0.5 | 0.5 | — | — | — | — | — |
| Octopirox⁴ | — | — | — | — | — | — | — | — | — | — | — | 0.2 | 0.2 | 0.5 | 0.5 |
| Fungus Testing | | | | | | | | | | | | | | | |
| NSA (12 days) | 3 | 0 | 0 | 0 | 0 | 0 | 0 | 0 | 0 | 0 | 4 | 0 | 2 | 0 | 1 |
| NSA (27 days) | | | 0 | 0 | | | | | | | | | | | |
| PDA (12 days) | 3 | 1 | 0 | 0 | 0 | 0 | 1 | 1 | 1 | 1 | 4 | 1 | 3 | 0 | 1 |
| PDA (27 days) | | | 0 | 0 | | | | | | | | | | | |
| At elevated pH | | | | | | | | | | | | | | | |
| NSA | 4 | 0 | 0 | 0 | 0 | 0 | 0 | 0 | 0 | 0 | 4 | 0 | 1 | 0 | 0 |
| PDA | 4 | 4 | 0 | 0 | 1 | 0 | 1 | 1 | 1 | 2 | 4 | 2 | 2 | 1 | 3 |

¹AgION AC 10D - a silver/copper zeolite antimicrobial agent from AgION Technologies, Inc., Wakefield, MA, USA
²Aqueous colloidal suspension of dichloro-octyl isothiazolone in water and ˜16% propylene glycol from Rohm & Haas, Philadelphia, PA, USA.
³Colloidal suspension of dichloro-octyl isothiazolone in phenyl ether and propylene glycol from Rohm & Haas, Philadelphia, PA, USA.
⁴Octopirox from Clariant Corporation of Charlotte, NC, USA.

and even more surprising that a synergy was found in bioefficacy with the aqueous based antifungal agent.

Though not intending to be bound by theory, it is believed that the synergy seen with the aqueous based fungicide was not seen with the non-aqueous counterpart due to the importance of water transport in the release mechanism for the antimicrobial active of the ion-exchange antimicrobial agent. It is thought that the phenyl ether solvent of the Rozone fungicide may have coated the particles of the antimicrobial agent or otherwise interfered with the transport mechanism, thereby preventing ion-release. Thus, the present invention is especially pertinent to fungicides that are aqueous based and/or have substantial water solubility. Alternatively, it may be necessary to treat the composition with additional surfactants to address whatever interaction may be present between the non-aqueous solvent and the ion-exchange antimicrobial agent.

### EXAMPLES 6-7 AND COMPARATIVE EXAMPLES CE11-CE14

Another series of experiments was conducted to evaluate the efficacy of combinations of the ion-exchange type antimicrobial agent with a myclobutanil fungicide (Immunox) or a chlorothalonil fungicide (Daconil—a substituted benzene fungicide) in preventing/inhibiting the growth Aspergillus niger. The formulations tested and the results attained therewith are shown in Table 2: the test levels employed were one-fourth (¼^{th}) the recommended use level. Each test sample was prepared and evaluated in accordance with the procedure set forth in the foregoing section (Examples 1-5). Samples of the untreated and treated filter paper were also evaluated as control examples (CE11 and CE12) in order to demonstrate the lack of inherent antifungal activity in the test paper as well as in the coating material itself, i.e., the vinyl acetate-acrylic copolymer.

Despite the low use levels of the active agents, as seen in Table 2, a marked synergy in antifungal bioefficacy was noted with the combination of the ion-exchange type antimicrobial agent and the myclobutanil fungicide (Immunox) in the nutrient rich NSA agar (Example 7). However, at the low levels tested, no change in bioefficacy was noted for those tests conducted in the highly nutritious potato dextrose agar. At the low levels tested for the combination with chlorothalonil fungicide (Daconil), there was no apparent benefit in either test environment. Because the chlorothalonil fungicide was completely effective against the A. niger in the NSA agar, even at the low level tested, it is believed that this active masked any benefit of the silver/copper zeolite. However, it is believed that a synergy would have manifested itself if lower levels of the chlorothalonil had been evaluated. Furthermore, again in light of what was seen with the olamine above, it is significant that the presence of the silver/copper zeolite antimicrobial did not adversely affect the antifungal performance of these fungicide actives.

TABLE 2

| | EXAMPLES | | | | | |
|---|---|---|---|---|---|---|
| | CE11^{a} | CE12^{b} | CE13 | 7 | CE14 | 8 |
| ACTIVE | | | | | | |
| AGENTS | | | | | | |
| AgION AC10D¹ | — | — | — | 1 | — | 1 |
| Immunox² | — | — | 0.2 | 0.2 | — | — |
| Daconil³ | — | — | — | — | 0.2 | 0.2 |
| Fungus Testing | | | | | | |
| NSA (12 days) | 4 | 4 | 4 | 1 | 0 | 0 |
| NSA (27 days) | 4 | 4 | 4 | 1 | 0 | 0 |
| PDA (12 days) | 4 | 4 | 4 | 4 | 4 | 4 |
| PDA (27 days) | 4 | 4 | 4 | 4 | 4 | 4 |

^{a}paper control
^{b}paper coated with vinyl resin control
¹AgION AC 10D - a silver/copper zeolite antimicrobial agent from AgION Technologies, Inc., Wakefield, MA, USA
²Spectrum Brands, St. Louis, MO, USA
³Syngenta Professional Products, Greensboro, NC, USA

Sarcomycetes Cervicae Studies

To further demonstrate the attributes of the present invention, several additional series of experiments were conducted in which the bioefficacy of certain combinations of an ion-exchange type antimicrobial agent with several different commercial fungicides was evaluated in relation to their ability to suppress and/or inhibit the growth of Sarcomycetes Cervicae (Fleishmann's Bakers yeast). Sarcomycetes Cervicae was selected as a model test organism as it is generally accepted in the industry as an indicator organism for a wide variety of fungi. In each of these experiments, the same general procedure as described below was followed unless otherwise indicated.

Experimental Detail: A growth medium was prepared by adding 10 grams of nutrient medium (Difco Sabouraud dextrose broth from BD of Franklin Lakes, N.J., USA) to 300 ml of distilled water. Fleishmann's Bakers yeast was then added to the growth medium while mixing using a magnetic stirrer until a uniform dispersion was obtained having an initial turbidity of between about 50 and 100 NTU as measured using a HF Instruments DRT 100B Turbidity Meter. Once the appropriate dispersion was obtained, 20 ml aliquots were then dispensed, with continued mixing, into 40 ml borosilicate glass vials with Teflon lined caps (VWR International Cat. No. 15900-004). Solutions/dispersions of the active agent(s) to be evaluated were then added to the vial and intimately shaken to ensure a good, substantially homogeneous mixture. The turbidity of each mixture was then determined and the vial transferred to an incubator maintained at 30° C. Each vial was periodically removed from the incubator and the mixture in the vials assessed for turbidity. The specific timing for such evaluation was asset forth in the discussion of the experiments and in the accompanying tables.

In reviewing the data in the following tables, it is to be appreciated that the limit of detection of the turbidity meter employed seemed to max out around 1100-1200 NTU. Thus, NTU values in this range merely indicate a high degree of turbidity which is reflective of yeast and, if present, the insoluble or poorly soluble additives; but, should not be taken as the actual turbidity or an indication that yeast growth had stopped. Ultimately, in assessing the data presented one should look for a low, initial delta NTU followed by a fairly similar, preferably non-increasing or slowly increasing delta NTU. As indicated in the tables below, the delta NTU may also be negative owing, it is believed, but not confirmed, to a disintegration or breaking down of the dead yeast and their proteins. Finally, as mentioned above, oftentimes the additive actives themselves added to the turbidity of the samples owing to their insolubility or poor solubility. The extent to which the additive actives contribute to the turbidity of the samples is most evident by comparing the turbidity of the control versus the examples and comparative examples at Time 0.

In each experiment, unless otherwise specified, a 2 ml aqueous solution containing the specified bioactive component or system was added to the 20 ml yeast suspension and mixed thoroughly. If used, surfactants were added separately in a concentrated solution in water: the volume added was negligible (a fraction of an ml). For convenience in understanding efficacy levels, the amounts or concentrations of the various components presented in each of the following tables and experiments are of the diluted material in the test vial: not of the concentration of the solution added to the test vial. Even so, concentrations presented are on the basis of a 20 ml total volume, not the actual 22+ ml volume. Multiplying each of the listed concentrations by 0.9 (or 0.95 with those compositions using 1 ml aqueous solutions) will provide a more accurate assessment of the concentration of the various components evaluated, i.e., 1% silver/copper zeolite composition is actually closer to 0.9% concentration. Finally, for those vials to which no bioactive system or component thereof was added (the controls) 2 ml of additional growth medium was added to ensure relative equivalent dilutions of the yeast.

In the tables below, the results are presented as the actual turbidity readings (NTU) with a sub-table presenting the change or delta in NTU values. Given the nature of the system, changes in turbidity are reflective of the relative performance/bioefficacy of the bioactive systems and their components. In certain instances, a high level of bioactive material, especially those that are substantially insoluble, e.g., the silver/copper zeolite, caused an immediate and relatively sharp increase in optical density or turbidity as compared to those without such constituents. Additionally, in those highly active systems, some of the immediate increase in turbidity was believed to have been a result of lysing of at least a portion on the yeast cells themselves. Consequently, especially in those examples having a high level of bioactive, it may be equally, if not more, important to look at the change in turbidity from the one or two hour turbidity results forward, not from time zero.

### EXAMPLES 9-13, COMPARATIVE EXAMPLES CE15-21

A first set of experiments evaluated the efficacy of a dithiocarbamate, specifically an ethylene(bis)dithiocarbamate (Mancozeb—manganese ethylene(bis)dithiocarbamate, DuPont Agricultural Products, Wilmington, Del.), in combination with the silver/copper zeolite (AgION AC10D, AgION Technologies, Inc., Wakefield, Mass.) in inhibiting the growth of Sarcomycetes Cervicae. The specific formulations tested and the results attained therewith are shown in Table 3. The amount of each ingredient is presented as the concentration in the vial: the numeral representing 10⁻⁰² wt percent. Thus, for example, Example 9 contained 0.0156% by weight AgION AC10D and 0.0313% by weight Mancozeb.

The synergy of Mancozeb with AgION AC10D is less clear; though it is believed present. As noted above, the turbidity meter employed seemed to max out at about 1100-1200 NTU. Thus, because these systems had such high initial turbidities to begin with (owing to the insoluble and poorly soluble actives), it was not possible to accurately report on yeast growth. For example, the turbidity owing to the high level of Mancozeb in Comparative Example 15 maxed out the turbidity meter so no readings on yeast growth was possible. Still, the data reflects a somewhat slower yeast growth in Examples 9 and 10 than was attained in any of the Comparative Examples. Notwithstanding the foregoing, the data clearly and unexpected showed that the addition of a conventional level of a surfactant, sodium lauroyl sarcosinate, markedly enhanced the bioefficacy of the combination (Example 13).

TABLE 3

| | EXAMPLES | | | | | | | | | | | |
|---|---|---|---|---|---|---|---|---|---|---|---|---|
| | CE15 | CE16 | CE17 | CE18 | CE19 | CE20 | CE21 | 9 | 10 | 11 | 12 | 13 |
| Active Agents | | | | | | | | | | | | |
| AgION | — | — | — | — | 3.13 | 1.56 | 0.781 | 1.56 | 0.781 | 0.391 | 0.391 | 0.781 |
| AC10D | | | | | | | | | | | | |
| Mancozeb | — | 6.25 | 3.75 | 1.25 | — | — | — | 3.13 | 1.88 | 1.25 | 0.938 | 1.88 |
| NaLS | — | — | — | — | — | — | — | — | — | — | — | 5 |
| Time (hours) | | | | | | | | | | | | |
| T0 | 46 | 1100 | 382 | 259 | 283 | 140 | 85 | 779 | 390 | 173 | 228 | 410 |
| T1 | 57 | 1081 | 573 | 260 | 282 | 148 | 95 | 790 | 418 | 184 | 235 | 385 |
| T18 | 1055 | 1200 | 1220 | 1118 | 930 | 940 | 975 | 1210 | 1095 | 1050 | 1105 | 443 |
| T24 | 1080 | 1218 | 1225 | 1120 | 985 | 960 | 985 | 1230 | 1085 | 1058 | 1103 | 511 |
| T48 | 1080 | 1260 | 1220 | 1160 | 1060 | 1006 | 980 | 1110 | 1070 | 1064 | 1120 | 1052 |
| Δ NTU | | | | | | | | | | | | |
| Tx − T0 | | | | | | | | | | | | |
| T18 | 1009 | 100 | 838 | 859 | 647 | 800 | 890 | 431 | 705 | 877 | 877 | 33 |
| T24 | 1034 | 128 | 843 | 861 | 702 | 820 | 900 | 451 | 695 | 885 | 875 | 101 |
| T48 | 1034 | 160 | 838 | 901 | 777 | 886 | 895 | 331 | 680 | 891 | 892 | 642 |

### EXAMPLES 14-17, COMPARATIVE EXAMPLES CE22-CE30

To further supplement the demonstrated benefit of the combination of a myclobutanil fungicide with the ion-exchange type antimicrobial agent as shown in Table 2 above, various dilutions of the silver/copper zeolite and Immunox (Spectrum Brands, St. Louis, Mo., USA) were evaluated on their own and in combination relative to their bioefficacy in preventing/inhibiting the growth of Sarcomycete Cervicae. The specific formulations and the results obtained with each were as set forth in Table 4. Once again, the results demonstrate the synergy of the combination of actives.

### EXAMPLES 18-19, COMPARATIVE EXAMPLES CE31-32

Following up on the prior set of examples, another series of experiments were conducted in the same manner as the preceding set of examples with the exception that the level of the ion-exchange type antimicrobial agent was held constant, at a level for which little antifungal activity had been shown in Table 4, while varying the amount of the Immunox fungicide. As indicated in Table 4, neither of these levels of Immunox fungicide showed even moderate antifungal properties against Sarcomycetes Cervicae when used by themselves. Surprisingly, however, as shown in Table 5, when combined with an otherwise ineffective or poorly effective level of the ion-exchange type antimicrobial agent, a significant degree in antifungal performance was manifested.

### EXAMPLES 21-26, COMPARATIVE EXAMPLES CE32-CE38

To further demonstrate the breadth of the present invention to combinations of the ion-exchange type antimicrobial agent and inorganic fungicides, especially non-transition metal-containing fungicides, a series of examples were prepared in which a combination of the silver/copper zeolite and sodium perborate tetrahydrate was evaluated for its effectiveness in preventing/inhibiting the growth of Sarcomycetes Cervicae. The specific formulations evaluated and the results attained therewith are presented in Table 6.

TABLE 4

| | Example | | | | | | | | | | | | |
|---|---|---|---|---|---|---|---|---|---|---|---|---|---|
| | CE22 | CE23 | CE24 | CE25 | CE26 | CE27 | CE28 | CE29 | CE30 | 14 | 15 | 16 | 17 |
| Active | | | | | | | | | | | | | |
| Agent | | | | | | | | | | | | | |
| AgION | — | — | — | — | — | 1:800 | 1:1600 | 1:3200 | 1:6400 | 1:800 | 1:1600 | 1:3200 | 1:6400 |
| AC10D | | | | | | | | | | | | | |
| Immunox | — | 1:64 | 1:128 | 1:256 | 1:512 | — | — | — | — | 1:64 | 1:128 | 1:256 | 1:512 |
| Time (hours) | | | | | | | | | | | | | |
| T0 | 64 | 48 | 57 | 54 | 54 | 1158 | 732 | 361 | 220 | 1061 | 641 | 339 | 217 |
| T1 | 73 | 54 | 57 | 61 | 66 | 1126 | 742 | 370 | 233 | 1035 | 635 | 339 | 222 |
| T18 | 686 | 483 | 580 | 652 | 629 | 1048 | 596 | 582 | 690 | 945 | 525 | 514 | 649 |
| T25 | 752 | 500 | 625 | 675 | 664 | 1034 | 555 | 638 | 750 | 918 | 500 | 591 | 700 |
| T42 | 841 | 584 | 701 | 810 | 814 | 984 | 491 | 670 | 823 | 849 | 436 | 660 | 810 |
| T66 | 776 | 632 | 786 | 774 | 776 | 928 | 427 | 654 | 764 | 727 | 376 | 649 | 793 |
| Δ NTU | | | | | | | | | | | | | |
| Tx − T1 | | | | | | | | | | | | | |
| T18 | 613 | 429 | 523 | 591 | 563 | −78 | −146 | 222 | 457 | −90 | −110 | 175 | 427 |
| T25 | 679 | 446 | 568 | 614 | 598 | −92 | −187 | 268 | 517 | −117 | −135 | 262 | 478 |
| T42 | 768 | 530 | 644 | 749 | 748 | −142 | −251 | 300 | 590 | −186 | −199 | 321 | 588 |
| T66 | 703 | 578 | 729 | 713 | 710 | −198 | −315 | 284 | 531 | −308 | −259 | 310 | 571 |

TABLE 5

| | |
|---|---|
| Active | Example |

| Agent | CE31 | 18 | 19 | 20 |
|---|---|---|---|---|
| AgION | — | 1:3200 | 1:3200 | 1:3200 |
| AC10D | | | | |
| Immunox | — | 1:64 | 1:128 | — |
| Time (hours) | | | | |
| T0 | 78 | 317 | 360 | 433 |
| T2 | 103 | 295 | 352 | 103 |
| T6 | 196 | 310 | 339 | 413 |
| T24 | 1028 | 575 | 679 | 942 |
| T48 | 1005 | 637 | 740 | 960 |
| T66 | 998 | 661 | 723 | 964 |
| Δ NTU Tx-T2 | | | | |
| T6 | 93 | 15 | −13 | −14 |
| T24 | 925 | 280 | 327 | 515 |
| T48 | 902 | 342 | 388 | 533 |
| T66 | 895 | 366 | 371 | 537 |

As seen in Table 6, the addition of an ineffective amount of the ion-exchange type antimicrobial agent (0.125% AgION AC10D) to varying amounts of the perborate fungicide markedly increased the efficacy of the latter: even to the point where a higher level of antifungal activity was achieved with less than one-tenth the amount of perborate shown (in this series of examples) to have the best antifungal performance (compare Comparative Example CE33 to Example 23. These examples demonstrate the synergistic antifungal properties when the two active agents are used in combination. Furthermore, these results demonstrate the economic and environmental benefit of the present invention in that less overall fungicide actives may be employed for excellent performance: a level of performance not achievable by either alone, at least not at the levels demonstrated.

Following on the foregoing, not only is there a synergy but the results in Table 6 seems to suggest that the presence of even a minor amount of the antimicrobial agent shifted the optimal level of the perborate in inhibiting yeast growth from 1% or more to something in the range of 0.25% to 0.1% or less. Thus, while there may be a desire to employ overkill, i.e., use higher levels of the actives than needed to ensure better and/or longer activity, surprisingly these results seem to suggest that one is better using the lower amounts of the actives as is now allowed and enabled by the present invention.

Although the present invention has been described with respect to the foregoing specific embodiments and examples, it should be appreciated that other embodiments utilizing the concept of the present invention are possible without departing from the scope of the invention. The present invention is defined by the claimed elements and any and all modifications, variations, or equivalents that fall-within the spirit and scope of the underlying principles.

TABLE 6

| | Example | | | | | | | | | | | | |
|---|---|---|---|---|---|---|---|---|---|---|---|---|---|
| | CE32 | CE33 | CE34 | CE35 | CE36 | CE37 | CE38 | 21 | 22 | 23 | 24 | 25 | 26 |
| Active Agent | | | | | | | | | | | | | |
| AgION AC10D | 0.125 | — | — | — | — | — | — | 0.125 | 0.125 | 0.125 | 0.125 | 0.125 | 0.125 |
| sodium | — | 1.0 | 0.5 | 0.25 | 0.1 | 0.05 | 0.025 | 1.0 | 0.5 | 0.25 | 0.1 | 0.05 | 0.025 |
| perborate | | | | | | | | | | | | | |
| tetrahydrate | | | | | | | | | | | | | |
| Time (hours) | | | | | | | | | | | | | |
| T0 | 120 | 83 | 99 | 90 | 92 | 94 | 96 | 372 | 456 | 469 | 450 | 462 | 431 |
| T1 | 215 | 102 | 106 | 106 | 105 | 109 | 120 | 297 | 375 | 398 | 500 | 522 | 438 |
| T24 | 872 | 58 | 99 | 235 | 379 | 465 | 516 | 284 | 300 | 311 | 336 | 319 | 336 |
| T44 | 1030 | 50 | 209 | 650 | 683 | 751 | 792 | 268 | 290 | 300 | 313 | 594 | 625 |
| T72 | 1020 | 46 | 968 | 684 | 695 | 769 | 795 | 266 | 291 | 309 | 310 | 768 | 695 |
| Δ NTU | | | | | | | | | | | | | |
| Tx − T1 | | | | | | | | | | | | | |
| T24 | 657 | −44 | −7 | 129 | 274 | 356 | 396 | −13 | −75 | −87 | −164 | −203 | −102 |
| T44 | 815 | −52 | 103 | 544 | 578 | 642 | 672 | −29 | −85 | −98 | −187 | 72 | 187 |
| T72 | 805 | −56 | 862 | 576 | 590 | 660 | 676 | −31 | −84 | −89 | −190 | 246 | 253 |

I claim:

1. An antifungal composition comprising a traditional antifungal agent in combination with one or more ion-exchange type antimicrobial agent.

2. The antifungal composition of claim 1 wherein the ion-exchange antimicrobial agent comprises an antimicrobial metal ion or metal ion source.

3. The antifungal composition of claim 2 wherein the antimicrobial metal-ion is selected from the group consisting of silver, copper, zinc, gold, mercury, tin, lead, iron, cobalt, nickel, manganese, arsenic, antimony, bismuth, barium, cadmium, chromium and thallium and combinations thereof.

4. The antifungal composition of claim 1 wherein the ion-exchange type antimicrobial agent comprising one or more ion-exchanged antimicrobial metal ions and an ion-exchange carrier therefore selected from the group consisting of zeolites, hydroxyapatites, zirconium phosphates and other ion-exchange ceramic materials.

5. The antifungal composition of claim 4 wherein the antimicrobial metal ion is selected from the group consisting of silver, copper, zinc, and gold and combinations of any two or more of the foregoing.

6. The antifungal composition of claim 4 wherein the antimicrobial metal ions is silver, alone or in combination with copper or zinc or both.

7. The antifungal composition of claim 1 wherein the antimicrobial agent is a zeolite having ion-exchanged silver ions, alone or in combination with copper ions or zinc ions or both.

8. The antifungal composition of claim 1 wherein the antifungal active is an amide, acyl amino acid, anilide, benzanilide, furanilide, sulfonanilide, bezamide, furamide, phenyl sulfamide, sulfonamide, valinamide, antibiotic, strobilurin, chloroneb, chlorothalonil, dichlorobenil, dichloran, PCNB, benzimidazole, benzothiazole, bridged diphenyl, carbamate, benzimidazoylcarbamate, carbanilate, conazole, imidazole, triazole, copper, dicarboximide, dichlorophenyl dicarboximide, phthalimide, dinitrophenol, dithiocarbamate, cyclic dithiocarbamate, polymeric dithiocarbamate, imidazole, inorganic mercuric, organomercury, morpholine, organophosphorus, organotin, oxathiin, oxazole, polysulfide, pyrazole, pyridine, pyrimidine, pyrrole, quinoline, quinone, quinoxaline, thiazole, thiocarbamate, thiophene, triazine, triazole, urea, or borate fungicide.

9. The antifungal composition of claim 1 wherein the antifungal active is a triazole, myclobutanil, imidazole, borate, isothiazolone, dithiocarbamate, and strobilurin fungicide.

10. The antifungal composition of claim 1 wherein the fungicide is an organic fungicide or a salt thereof.

11. The antifungal composition of claim 1 wherein the fungicide is a borate.

12. The antifungal composition of claim 1 wherein the fungicide is not a copper-based fungicide.

13. The antifungal composition of claim 1 wherein the fungicide is not a zinc based fungicide.

14. The antifungal composition of claim 1 wherein the antifungal agent is aqueous based or substantially water soluble.

15. The antifungal composition of claim 1 further comprising a binder selected from the group consisting of hydrophilic polymers, thermoset resins, thermoplastic polymer and silicates.

16. The antifungal composition of claim 1 wherein each of the two actives is present in an amount that is conventional for its use and the level of antifungal activity is at least as high as would be in the absence of the antimicrobial agent.

17. The antifungal composition of claim 1 wherein each of the two actives is present in an amount that is conventional for its use and the level of antifungal activity is higher than would be in the absence of the antimicrobial agent.

18. The antifungal composition of claim 1 wherein each of the two actives is present in an amount that is less than conventional for its use.

19. The antifungal composition of claim 1 wherein each of the two actives is present in an amount that, when used on their own, is ineffective or poorly effective for preventing the growth of mold.

20. The antifungal composition of claim 1 wherein the fungicide is a complex or combination of two or more fungicide actives.
